(19) United States
(12) Patent Application Publication
(10) Pub. No.: US 2018/0008768 A1
(43) Pub. Date: Jan. 11, 2018

(54) Mobile Infusion Pump

(71) Applicant: Raumedic AG, Munchberg (DE)

(72) Inventors: Jörg Prescher, Ramsthal (DE); Daniel Moreth, Hof (DE); Björn Gläsel, Kirchenlamitz (DE)

(21) Appl. No.: 15/646,335

(22) Filed: Jul. 11, 2017

(30) Foreign Application Priority Data
Jul. 11, 2016 (DE) 10 2016 212 579.6

(51) Int. Cl. A61M 5/148

(52) U.S. Cl. CPC A61M 5/148; A61M 2205/3334

(57) ABSTRACT

A mobile infusion pump is used for the constant delivery of a fluid medium. The infusion pump comprises a balloon body for receiving the medium as well as a filling unit and a delivery unit that are in a fluid connection with the balloon body for filling the medium into the balloon body and delivering the medium from the balloon body to a patient. A throttling unit defines a delivery flow rate of the medium. By means of a plurality of continuous longitudinal ribs arranged on a part of the balloon body configured as a tube body, the expansion behavior of the balloon body is improved.

### CROSS-REFERENCE TO RELATED APPLICATIONS

This application claims priority of German Patent Application Serial No. 10 2016 212 579.6 filed on Jul. 11, 2016, pursuant to 35 U.S.C. (a)-(d), the content of which is incorporated herein by reference in its entirety as if fully set forth herein.

### FIELD OF THE INVENTION

The invention relates to a mobile infusion pump for the constant delivery of a fluid medium. The invention further relates to a balloon body for an infusion pump of this type and to a tube body as a semi-finished product to be used as a component of a balloon body of this type.

### BACKGROUND OF THE INVENTION

Mobile infusion pumps are used in ambulant medical treatment, in particular in the fields of parenteral nutrition, chemotherapy and pain management. Conventional infusion pumps are equipped with a balloon body for receiving the fluid medium. The balloon body is filled with the fluid medium by means of a filling unit, causing the balloon body to expand. The expansion of the balloon body generates restoring forces in said balloon body, which allow the fluid medium to be delivered to a patient under pressure. Delivery is carried out using a delivery unit, which forms a fluid connection between the infusion pump and a patient. Infusion pumps of this type are for instance disclosed in the specifications U.S. Pat. No. 8,398,595 B2, U.S. Pat. No. 7,341,572 and U.S. Pat. No. 8,439,862.

### SUMMARY OF THE INVENTION

Therefore, the present invention is based on the object of providing an infusion pump having a precise dosage behavior during the delivery of the fluid medium. In particular, a variation of the total time of the delivery process shall amount to no more than 10% of a desired delivery time.

This object is achieved according to the invention by a mobile infusion pump for the constant delivery of a fluid medium, the mobile infusion pump comprising a balloon body for receiving the medium; a filling unit, which is in fluid connection with an internal volume of the balloon body, for filling the medium into the balloon body; and a delivery unit, which is in fluid connection with an internal volume of the balloon body, for delivering the medium from the balloon body, wherein the delivery unit is provided with a throttling unit for defining a delivery flow rate of the medium; and a part of the balloon body is a tube body with a cross-sectional profile comprising a plurality of continuous longitudinal ribs.

The mobile infusion pump according to the invention comprises a balloon body for receiving the medium, a filling unit and a delivery unit. The filling unit and delivery unit are in a fluid connection with an internal volume of the balloon body to fill the fluid medium into the balloon body and deliver the fluid medium from the balloon body to a patient. In order to define a delivery flow rate of the fluid medium, the delivery unit is further provided with a throttling or fluid restricting unit. In order to optimize the expansion behavior of the balloon body, and therefore the dosage behavior of the infusion pump, it is intended for a part of the balloon body to be configured as a tube body having a cross-sectional profile with a plurality of continuous longitudinal ribs. The longitudinal ribs increase the stability of the tube body. The expansion behavior can therefore be controlled in a targeted manner as only individual segments disposed between the longitudinal ribs are able to expand. The controlled expansion behavior of the balloon body ensures a constant delivery pressure, which allows the dosage behavior and the time of the delivery process to be defined more precisely. An unwanted varying expansion of the balloon body, which would result in a varying delivery pressure, is thus avoided. Correspondingly, there is no unwanted variation of the delivery time.

The increased stability of the tube body further has the advantage of an easier manipulation and arrangement of the balloon body in the infusion pump.

Configuring at least one of the longitudinal ribs as an internal longitudinal rib forming part of an internal profile of the tube body allows the contact of the balloon body with a support body of the infusion pump to be controlled in a targeted manner. This prevents the balloon body from adhering to the support body, which may occur when the infusion pump is stored for a longer period of time.

When at least two internal longitudinal ribs are provided, this ensures an improved distribution of the support forces of the internal longitudinal ribs along an internal profile of the tube body. Furthermore, the contact surface of the tube body with the support body is reduced even more. During delivery, a part of the fluid medium may remain in the compartments between the longitudinal ribs, which may prevent a complete emptying of the balloon body.

The embodiment in which an external profile of the tube body is produced by at least one longitudinal rib configured as an external longitudinal rib forming part of an external profile of the tube body has the advantage that the stability of the tube body is increased without preventing a complete emptying of the balloon body during the delivery of the fluid medium.

Providing a plurality of external longitudinal ribs, in particular at least two, in particular at least four external longitudinal ribs, improves the distribution of the support forces along an outer circumference of the tube body.

According to a preferred embodiment, an infusion pump is provided that combines at least one internal longitudinal rib forming part of an internal profile of the tube body and at least one external longitudinal rib forming part of an external profile of the tube body. The internal and external longitudinal ribs are arranged on the same level when seen in the circumferential direction so as to increase the stability of the respective longitudinal ribs. The support forces of the longitudinal ribs are therefore increased locally.

According to another preferred embodiment, an infusion pump is provided that combines at least one internal longitudinal rib forming part of an internal profile of the tube body and at least one external longitudinal rib forming part of an external profile of the tube body, too, with the at least one internal longitudinal rib and the at least one external longitudinal rib being arranged such as to be staggered relative to each other when seen in the circumferential direction of the tube body. Said staggered arrangement of the internal and external longitudinal ribs when seen in the circumferential direction ensures an optimal distribution of the support forces.

The invention further relates to a balloon body for an infusion pump as outlined above. Another subject matter of the invention is a tube body as a semi-finished product to be used as a component of a balloon body of this type.

An exemplary embodiment of the invention will hereinafter be explained in more detail with reference to the drawing.

### BRIEF DESCRIPTION OF THE DRAWING

FIG. 1 shows a schematic view of a mobile infusion pump for the constant delivery of a fluid medium, and

FIGS. 2 to 5 show cross-sectional views of alternative embodiments of a tube body as a component of a balloon body to be used in the infusion pump.

### DESCRIPTION OF THE PREFERRED EMBODIMENT

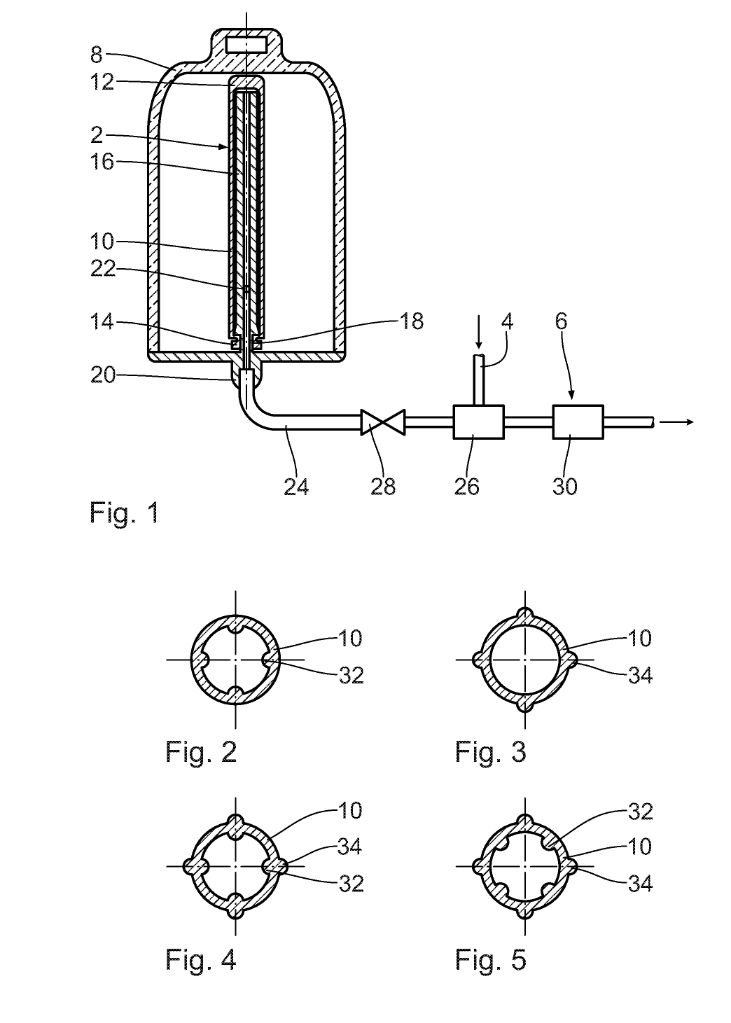

FIG. 1 shows a mobile infusion pump for the constant delivery of a fluid medium. The infusion pump comprises a balloon body 2 for receiving the fluid medium, a filling unit 4 and a delivery unit 6. In order to fill and deliver the fluid medium, the filling unit 4 and the delivery unit 6 are in another fluid connection, described below, with an internal volume of the balloon body 2.

In order to protect the balloon body 2 from external influences, the balloon body 2 is surrounded by a solid external housing 8. The external housing 8 is transparent, thus allowing the fill level of the balloon body 2 to be monitored at any time.

The balloon body 2 comprises a tube body 10, a closed end portion 12 and a collar 14 arranged opposite the end portion 12. The balloon body 2 encloses a cylindrical support body 16 the longitudinal axis 17 of which coincides with the longitudinal axis of the tube body 10. The collar 14 of the balloon body 2 surrounds a tapered neck portion 18 of the support body 16. In other words, the support body 16 is disposed entirely inside the internal volume of the balloon body 2. The collar 14 ensures that the internal volume of the balloon body 2 is sealed with respect to the remaining housing and the balloon body 2 is held on the support body 16. The support body 16 is connected to the external housing 8. A tube connector 20 is arranged on the external housing as an extension of the support body 16. The support body 16 is configured as a hollow body that is in fluid connection with the tube connector 20. A fluid connection between the hollow body of the support body 16 and the internal volume of the balloon body 2 is provided by radial holes 22 through the cylindrical shell of the support body 16.

In the exemplary embodiment shown, the tube connector is provided with a tube 24 forming part of the fluid connection between the internal volume of the balloon body 2 and the filling unit 4 or the delivery unit 6, respectively. A coupling 26 is arranged at the end of the tube 24 opposite the tube connector 20. By means of the coupling 26, a fluid connection can be formed selectively between the tube 24 and the filling unit 4 for filling in the fluid medium or between the tube 24 and the delivery unit 6 for delivering the fluid medium. The tube 24 is provided with a valve 28 for shutting off the fluid connection. The valve 28 may in particular be configured as a tube clamp. In other words, the fluid connection between the internal volume of the balloon body 2 and the filling unit 4 or delivery unit 6, respectively, comprises the coupling 26, the tube 24 comprising the valve 28, the tube connector 20 and the support body 16 configured as a hollow body. As an alternative to the embodiment shown here comprising a single tube 24 and a coupling 26, it is possible as well to provide separate fluid connections for the filling unit 4 and the delivery unit 6.

The filling unit 4 is not shown in more detail in FIG. 1. The filling unit 4 may for instance be configured as a syringe connected via a syringe connector by means of which the fluid medium is injected into the balloon body 2. As an alternative thereto, different filling units may be provided as well.

The delivery unit 6 arranged downstream of the coupling 26 comprises a throttling unit 30 for defining a delivery flow rate of the fluid medium. The rest of the delivery unit 6 is not shown explicitly in FIG. 1. It comprises a fluid connection towards the patient, which is for instance configured as an intramuscular line.

The balloon body 2 is made of an elastic material. In the empty state, the internal volume of the balloon body 2 is minimal. The internal volume is however at least equal to the volume of the support body 16. When filling in the fluid medium, the fluid medium flows through the radial hole 22 of the support body 16 into the internal volume of the balloon body 2, causing the balloon body 2 to expand. When the filling process is finished, the internal volume of the balloon body 2 has expanded by the volume of the fluid medium received therein. The volume of the fluid medium received therein may be adapted to the treatment and requirements of the patient. It may for instance amount to 75 ml or 100 ml.

When the elastic material of the balloon body 2 expands, this generates a restoring force that serves as a pumping pressure when delivering the fluid medium. In order to generate a pumping pressure that is as constant as possible during the entire delivery time, it is necessary for the balloon body 2 to expand constantly when filling in the fluid medium. In order to ensure an expansion of the balloon body 2 that is as constant as possible, the tube body 10 of the balloon body 2 is provided with longitudinal ribs. The longitudinal ribs run continuously along the longitudinal axis of the tube body 10 along the entire length thereof. The longitudinal ribs stabilize the tube body 10 along a longitudinal axis. This allows only individual segments between the longitudinal ribs to be expanded, which improves the expansion behavior of the balloon body 2 and, consequently, the dosage behavior of the infusion pump.

FIGS. 2 to 5 show alternative embodiments of tube bodies 10 with various arrangements of longitudinal ribs. FIGS. 2 to 5 show cross-sectional views thereof in a direction perpendicular to the longitudinal axis of the different tube bodies 10.

FIG. 2 shows a tube body 10 provided with four longitudinal ribs, the longitudinal ribs being configured as internal ribs 32 forming part of an internal profile of the tube body 10. Along with an increased stability of the tube body 10, this has the advantage that a contact surface of the internal profile of the tube body 10 with the support body 16 is minimized. This may prevent the tube body 10 from adhering to the support body 16. Such adherence may adversely affect the expansion behavior of the balloon body 2. In the alternative embodiment comprising internal longitudinal ribs shown in FIG. 2, a residual amount of the fluid medium remains in the compartments between the internal longitudinal ribs 32 during the delivery of the fluid medium and is not delivered to the patient. In order to avoid this, it is conceivable to provide longitudinal ribs as shown in FIG. 3, said longitudinal ribs being configured as external longitudinal ribs 34 forming part of an external profile of the tube body 10.

The stability and the expansion behavior of the balloon body 2 can be improved even more by combining internal longitudinal ribs 32 and external longitudinal ribs 34. As shown in FIG. 4, the internal longitudinal ribs 32 may be arranged on a level with the external longitudinal ribs 34 when seen in the circumferential direction of the tube body 10. The external longitudinal ribs 34 and internal longitudinal ribs 32 arranged opposite to each other reinforce one another in such a way that the stability of the tube body 10 along the longitudinal ribs is increased. As an alternative thereto, the internal longitudinal ribs 32 and the external longitudinal ribs 34 may be arranged such as to be staggered relative to each other when seen in the circumferential direction 10 of the tube body 10 (FIG. 5). This results in an even better distribution of the support forces of the longitudinal ribs and an even more optimized expansion behavior of the balloon body.

1. A mobile infusion pump for the constant delivery of a fluid medium, the mobile infusion pump comprising:
a balloon body for receiving the medium;
a filling unit, which is in fluid connection with an internal volume of the balloon body, for filling the balloon body with the medium; and
a delivery unit, which is in fluid connection with an internal volume of the balloon body, for delivering the medium from the balloon body, wherein the delivery unit is provided with a throttling unit for defining a delivery flow rate of the medium; and
wherein a part of the balloon body comprises a tube body with a cross-sectional profile comprised of a plurality of continuous longitudinal ribs.

2. The infusion pump according to claim 1, wherein at least one of the longitudinal ribs is configured as an internal longitudinal rib forming part of an internal profile of the tube body.

3. The infusion pump according to claim 2, wherein at least two internal longitudinal ribs are provided.

4. The infusion pump according to claim 1, wherein at least one of the longitudinal ribs is configured as an external longitudinal rib forming part of an external profile of the tube body.

5. The infusion pump according to claim 4, wherein at least two external longitudinal ribs are provided.

6. The infusion pump according to claim 5, wherein at least four external longitudinal ribs are provided.

7. The infusion pump according to claim 1, wherein at least one internal longitudinal rib is provided as part of an internal profile of the tube body and at least one external longitudinal rib is provided as part of an external profile of the tube body, wherein the at least one internal longitudinal rib is arranged on a level with the at least one external longitudinal rib when seen in a circumferential direction of the tube body.

8. The infusion pump according to claim 1, wherein at least one internal longitudinal rib is provided as part of an internal profile of the tube body and at least one external longitudinal rib is provided as part of an external profile of the tube body, the at least one internal longitudinal rib and the at least one external longitudinal rib being arranged such as to be staggered relative to each other when seen in the circumferential direction of the tube body.

9. The infusion pump according to claim 1, wherein the balloon body is elongated and tubular.

10. The infusion pump according to claim 1, wherein the tube body comprises a semi-finished product to be used as a component of the balloon body.

11. A mobile infusion pump for the constant delivery of a fluid medium, the mobile infusion pump comprising:
a balloon comprised of a tubular body defining an internal volume, the tubular body of the balloon comprised of a plurality of generally longitudinally extending ribs;
a filling unit in fluid flow communication with the balloon, the filling unit configured for filling at least part of the internal volume of the balloon with fluid medium; and
a delivery unit comprised of a throttling unit in fluid flow communication with the balloon, the delivery unit configured for receiving fluid medium from the internal volume of the balloon and outputting the fluid medium at a delivery flow rate defined by the throttling unit.

12. The infusion pump according to claim 11, wherein at least one of the ribs is an elongated external rib that extends generally longitudinally exteriorly along an external surface of the balloon.

13. The infusion pump according to claim 11, wherein the balloon has a plurality of spaced apart generally longitudinally extending external ribs.

14. The infusion pump according to claim 11, wherein the balloon has four circumferentially spaced apart elongate and generally longitudinally extending external ribs.

15. The infusion pump according to claim 11, wherein at least one of the ribs is an elongated internal rib that extends generally longitudinally interiorly along an internal surface of the balloon.

16. The infusion pump according to claim 11, wherein the balloon has a plurality of spaced apart generally longitudinally extending internal ribs.

17. The infusion pump according to claim 11, wherein the balloon further comprises (a) at least one generally longitudinally extending internal rib, and (b) at least one generally longitudinally extending external rib, and wherein each at least one external rib is radially outwardly spaced from and generally axially overlies a corresponding at least one internal rib.

18. The infusion pump according to claim 11, wherein the balloon further comprises (a) at least one generally longitudinally extending internal rib, and (b) at least one generally longitudinally extending external rib, and wherein each at least one external rib is circumferentially spaced from a corresponding at least one internal rib.
